(19) United States
(12) Patent Application Publication
(10) Pub. No.: US 2026/0029368 A1
(43) Pub. Date: Jan. 29, 2026

(54) ELECTROCHEMICAL GAS SENSOR AND CORRESPONDING METHOD

(71) Applicant: Testo SE & Co. KGaA, Titisee-Neustadt (DE)

(72) Inventors: Johannes DORNHOF, Kirchzarten (DE); Gregor STEINER, Titisee-Neustadt (DE); Reinhold MÜNCH, Freiburg (DE)

(73) Assignee: Testo SE & Co. KGaA, Titisee-Neustadt (DE)

(21) Appl. No.: 19/266,372

(22) Filed: Jul. 11, 2025

(30) Foreign Application Priority Data
Jul. 29, 2024 (EP) 24191532.1

(51) Int. Cl. G01N 27/413; G01N 33/00

(52) U.S. Cl. CPC G01N 27/413; G01N 33/0027

(57) ABSTRACT

In the case of an electrochemical gas sensor, it is provided to change a working voltage applied to at least two electrodes (2), in such a way that a potential drift on at least one electrode (2) of the gas sensor (1) is compensated for.

### CROSS-REFERENCE TO RELATED APPLICATIONS

This application claims priority from European Patent Application No. 24191532.1, filed Jul. 29, 2024, which is incorporated herein by reference as if fully set forth.

### TECHNICAL FIELD

The invention relates to an electrochemical sensor having at least two electrodes and having a voltage source with which a working voltage is able to be applied between the at least two electrodes.

The invention further relates to a method for operating an electrochemical gas sensor, a working voltage being applied between at least two electrodes.

### BACKGROUND

Electrochemical gas sensors are known and are used, for example, to detect or determine a presence and/or a quantity of a gas.

For this purpose, electrochemical gas sensors are typically filled with an electrolyte in which a plurality of electrodes are immersed, predetermined redox reactions taking place on the electrodes in the electrolytes via an applied potential difference, which reactions lead to a measurable current flow.

In this case, a working point of the electrochemical gas sensor, which results from the desired redox reaction, the electrodes used and the electrolyte, is established by setting a working voltage.

It has turned out that the working point of electrochemical gas sensors can shift unnoticed, e.g. if too high a concentration of the analyte or else unwanted substances come into contact with the electrodes and can thus trigger unwanted side reactions.

### SUMMARY

The invention is concerned with reducing or eliminating disturbing influences on the operation of an electrochemical gas sensor in a simple manner.

To achieve the stated object, one or more of the features disclosed herein are provided in the case of an electrochemical gas sensor. In particular, to achieve the stated object in the case of an electrochemical gas sensor of the type described at the outset, it is thus proposed that an adjustment device is formed, with which a voltage value of the working voltage is able to be changed. The invention makes use of the knowledge that contamination of such an electrochemical gas sensor by unwanted substances leads to a potential shift on at least one of the at least two electrodes, which potential shift is able to be compensated for or at least reduced by a change in the working voltage. It is thus possible, in a simple manner, to avoid contamination of the gas sensor leading to inadmissibly deviating measurement results.

In one configuration of the invention, provision can be made for one electrode of the at least two electrodes to be a sensing electrode. In this case, it is advantageous that a change in the potential level of the reference electrode is able to be compensated for in a simple manner.

In one configuration of the invention, provision can be made for one electrode of the at least two electrodes to be a reference electrode. In this case, it is advantageous that a reference point which is not normally affected by external disturbance variables is able to be used.

In one advantageous configuration of the invention, provision can be made for a counter electrode and a current measuring device to be formed, the current measuring device being able to be used to measure a current of a current flow between the measuring electrode and the counter electrode. An electrical measurement signal which correlates with the redox reaction occurring on the sensing electrode and/or the counter electrode is thus able to be generated in a simple manner.

In one advantageous configuration, provision can be made for a potential measuring device to be formed, with which a potential level of one electrode of the at least two electrodes is able to be measured. In this case, it is advantageous that a change in a potential of an electrode is able to be carried out in a simple manner. For example, this electrode can be the reference electrode already mentioned and/or the counter electrode already mentioned.

In one advantageous configuration, provision can be made for a potential measuring device, in particular the potential measuring device already mentioned, to be set up to measure a voltage between a or the reference electrode and a or the counter electrode. A simple means for detecting a change in a potential level of an electrode is thus provided.

In one configuration of the invention, provision can be made for a control device to be formed, with which a voltage value of the working voltage is able to be predefined as a function of a potential level of an electrode, in particular of the potential level already mentioned. In this case, it is advantageous that a potential drift is able to be at least partially compensated for automatically. Preferably, this electrode is the reference electrode.

In one configuration of the invention, provision can be made for the control device to have a constant current source. The working voltage is thus able to be provided in a defined and simple manner.

In one advantageous configuration, provision can be made for the control device to have a variable resistor. Adjustability of the working voltage is thus able to be achieved in a simple manner.

In one advantageous configuration, provision can be made for the working voltage to be determined by a voltage drop across a, for example the mentioned, variable resistor.

Alternatively, the working voltage can be formed in other ways, for example also by a variable current source, for example an operational amplifier.

To achieve the stated object, one or more of the features directed to a method, are provided in the case of a method. In particular, to achieve the object in the case of a method of the type described at the outset, the invention thus proposes that the working voltage is changed to compensate for a change in at least one potential level of at least one electrode of the at least two electrodes. A method is thus described, which can be easily automated, for example by virtue of the change in the working voltage being determined automatically from the change in the potential level. Particularly advantageously, the electrode on which the potential level is changed is the reference electrode. However, the sensing electrode can also be used for this purpose or both.

In one configuration of the invention, provision can be made for the working voltage to be changed if the change in the potential level exceeds a predetermined threshold value. In this case, it is advantageous that the number of changes in the working voltage is able to be limited to a small amount. For example, the threshold value can be selected such that in the event of a potential drift beyond the threshold value, lasting damage to the electrochemical gas sensor is to be feared or a measurement characteristic of the electrochemical gas sensor is unacceptably deformed.

In one configuration of the invention, provision can be made for the change in the potential level to be ascertained from a voltage difference between an electrode, which is preferably the reference electrode, and a counter electrode. A simple way of determining the potential drift is thus able to be provided.

In one configuration of the invention, provision can be made for the working voltage to be varied such that a calculated voltage difference between a sensing electrode, for example the sensing electrode already mentioned, and a counter electrode, for example the counter electrode already mentioned, lies within a (predefinable or predefined) voltage-value window. An automatable criterion according to which the working voltage is able to be corrected is thus predefined. It is particularly favorable here if the working voltage is varied such that the calculated voltage difference is equal to a default value.

A calculated voltage difference can be characterizable, for example, in that it results from an addition, with the correct algebraic sign, of measured voltages.

### BRIEF DESCRIPTION OF THE DRAWINGS

The invention will now be described in more detail with reference to an exemplary embodiment, but is not restricted to this exemplary embodiment.

In the drawings:

FIG. 1 shows an exploded view of an electrochemical gas sensor,

FIG. 2 shows a schematic circuit diagram of an electrochemical gas sensor,

FIG. 3 shows a schematic illustration of potential level with associated voltage differences, and

FIG. 4 shows one possible realization of a variable voltage source of the circuit according to FIG. 2.

### DETAILED DESCRIPTION

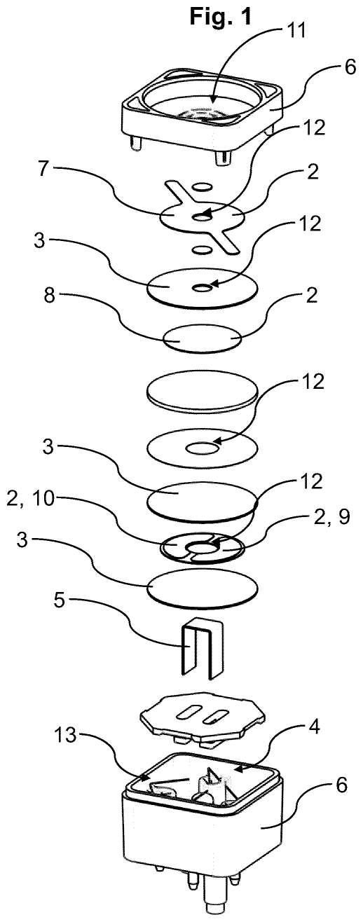

In the example of an electrochemical gas sensor 1 according to the invention shown in FIG. 1, four electrodes 2 are provided, between which electrolyte-impregnated membranes 3 are arranged. This electrolyte is supplied in a store 4 via a wick 5 to the membranes 3.

Here, the store 4 is arranged in the housing 6 which also accommodates the electrodes 2 and membranes 3.

Here, the electrodes 2 are in the form of a sensing electrode 7, auxiliary electrode 8, counter electrode 9 and reference electrode 10.

Via a gas path 11, which is indicated by openings 12, a measuring gas is supplied from the outside to the electrodes 2 which are in contact with the electrolyte provided by the membranes 3. This leads to a redox reaction of which one reaction direction occurs at the sensing electrode 7 and the opposite reaction occurs at the counter electrode 9.

To set a working point, a working voltage is applied between the sensing electrode 7 and the reference electrode 10.

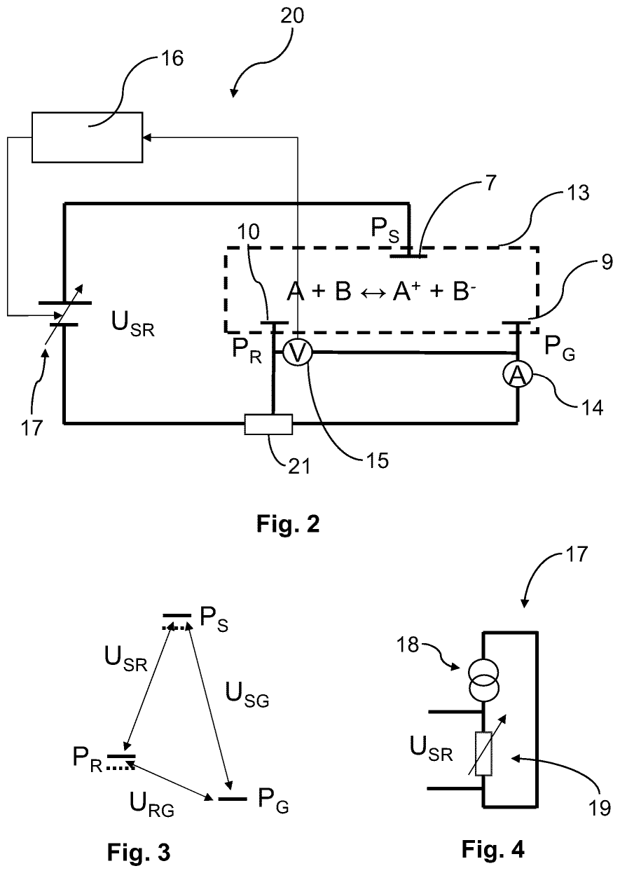

FIG. 2 shows a schematic circuit diagram of the electrochemical gas sensor 1.

In the electrolyte 13, the redox reaction

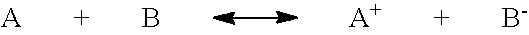

occurs.

All electrodes are immersed in the electrolyte 13.

Due to the redox reaction, a current flows between the sensing electrode 7 and the counter electrode 9, which current is able to be measured using a current measuring device 14.

In order that the desired reaction occurs, a working voltage U_{SR} is applied between the sensing electrode 7 and the reference electrode 10.

FIG. 3 shows the schematic illustration of the potential levels P_{S}, P_{G}, P_{R} at the sensing electrode (S), counter electrode (G) and reference electrode (R).

If a shift in the potential P_{R} at the reference electrode 10 is caused by contamination, the working voltage U_{SR} is updated in order that the potential difference U_{SG} between the sensing electrode 7 and the counter electrode 9 remains at a desired value.

In order to detect a drift in the potentials, provision can be made, for example, for a voltage difference between the potential P_{G} of the counter electrode 9 and the potential P_{R} of the reference electrode 10 to be measured.

The diagram immediately reveals that the potential difference or calculated voltage difference is U_{SG}=U_{SR}+U_{RG}.

Therefore, if this voltage difference U_{RG} is measured using a potential measuring device 15, it is possible to vary the working voltage U_{SR} for compensation purposes.

FIG. 2 shows a simplified schematic circuit diagram for explaining the electrochemical conditions. The actual wiring differs from this in a known manner.

From FIG. 2 it can be seen that the potential measuring device 15 is set up to measure a voltage U_{RG} between the reference electrode R and the counter electrode G.

An adder 21 conveys the potentials P_{R} and the voltage source U_{SR} to the counter electrode 9.

The signal of the potential measuring device 15 controls a control device 16. This control device 16 generates a control signal for the variable voltage source 17 as a function of the input signal.

This control occurs in such a way that a potential difference U_{SG} between the sensing electrode 7 and the counter electrode 9 remains constant (e.g. −1150 mV) or at least lies within a predefined voltage window.

In FIG. 4 it can be seen that the variable voltage source is formed from a fixed current source 18 and a variable resistor 19, the working voltage U_{SR} being tapped across the resistor 19.

The control signal of the control device 16 varies the resistance value of the resistor 19.

The potential measuring device 15, the control device 16 and the variable voltage source 17 form one example of an adjustment device 20 for the working voltage.

FIG. 3 explains the principle of the method according to the invention.

A working voltage U_{SR} is applied between the sensing electrode 7 and the reference electrode 10.

If the potential level of the reference electrode 10 changes, this being illustrated by a dotted line, the potential level of the sensing electrode 10 would also move. This change is detected, and the working voltage U_{SR} is changed by the same amount of the potential shift, but with an inverse sign, such that the calculated voltage difference U_{SG} remains unchanged. In one variant of the method, this correction can only be triggered if the potential drift exceeds a predetermined threshold value.

In the case of an electrochemical gas sensor 1, it is thus proposed to change a working voltage applied to at least two electrodes 2, in such a way that a potential drift on at least one electrode 2 of the gas sensor 1 is compensated for.

### LIST OF REFERENCE SIGNS

1 electrochemical gas sensor
2 electrode
3 membrane
4 store
5 wick
6 housing
7 sensing electrode
8 auxiliary electrode
9 counter electrode
10 reference electrode
11 gas path
12 opening
13 electrolyte
14 current measuring device
15 potential measuring device
16 control device
17 voltage source
18 constant current source
19 resistor
20 adjustment device
21 adder

1. An electrochemical gas sensor (1), comprising:
at least two electrodes (2);
a voltage source (17) with which a working voltage is adapted to be applied between the at least two electrodes (2); and
an adjustment device (20) with which a voltage value of the working voltage is changeable.

2. The electrochemical gas sensor (1) as claimed in claim 1, wherein one said electrode (2) of the at least two electrodes (2) is a sensing electrode (7).

3. The electrochemical gas sensor (1) as claimed in claim 2, wherein one said electrode (2) of the at least two electrodes (2) is a reference electrode (10).

4. The electrochemical gas sensor (1) as claimed in claim 3, further comprising a counter electrode (9) and a current measuring device (14), the current measuring device (14) being adapted to measure a current of a current flow between the sensing electrode (7) and the counter electrode (9).

5. The electrochemical gas sensor (1) as claimed in claim 1, further comprising a potential measuring device (15), with which a potential level of one said electrode (2) of the at least two electrodes (2) is adapted to be measured.

6. The electrochemical gas sensor (1) as claimed in claim 5, further comprising a counter electrode (9), wherein one said electrode (2) of the at least two electrodes (2) is a sensing electrode (7), another said electrode (2) of the at least two electrodes (2) is a reference electrode (10), and the potential measuring device (15), and the potential level of at least one of the reference electrode (10) or of the counter electrode (9) is adapted to be measured.

7. The electrochemical gas sensor (1) as claimed in claim 6, wherein the potential measuring device (15) is set up to measure a voltage between the reference electrode (10) and the counter electrode (9).

8. The electrochemical gas sensor (1) as claimed in claim 1, further comprising a control device (16) that is adapted to define a voltage value of the working voltage as a function of the potential level of one said electrode (2).

9. The electrochemical gas sensor (1) as claimed in claim 8, further comprising a counter electrode (9), wherein one said electrode (2) of the at least two electrodes (2) is a sensing electrode (7), another said electrode (2) of the at least two electrodes (2) is a reference electrode (10), and wherein the control device (16) is adapted to define the voltage value of the working voltage as a function of the potential level of at least one of the reference electrode (10) or the counter electrode (9).

10. The electrochemical gas sensor (1) as claimed in claim 8, wherein the control device (16) has a constant current source (18).

11. The electrochemical gas sensor (1) as claimed in claim 8, wherein the control device (16) has a variable resistor (19).

12. The electrochemical gas sensor (1) as claimed in claim 11, wherein the working voltage is determined by a voltage drop across the or a variable resistor (19).

13. A method for operating an electrochemical gas sensor (1), the method comprising:
applying a working voltage between at least two electrodes (2), and
changing the working voltage to compensate for a change in a potential level of one electrode (2) of the at least two electrodes (2).

14. The method as claimed in claim 13, wherein the working voltage (U_{SR}) is changed if a change in the potential level exceeds a predetermined threshold value.

15. The method as claimed in claim 14, further comprising ascertaining the change in the potential level from a voltage difference between one of the at least two electrodes (2) and a counter electrode (9).

16. The method as claimed in claim 15, wherein the working voltage is varied such that a calculated voltage difference between a sensing electrode (7) and the counter electrode (9) lies within a voltage-value window.

17. The method as claimed in claim 16, wherein the working voltage is varied such that the calculated voltage difference between the sensing electrode (7) and the counter electrode (9) is equal to a default value.
